(19) United States
(12) Patent Application Publication
(10) Pub. No.: US 2002/0065241 A1
(43) Pub. Date: May 30, 2002

(54) Antigenic peptide concatomers

(76) Inventor: Srinivas Shankara, Shrewsbury, MA (US)

Correspondence Address: Deborah A. Dugan, Genzyme Corporation, 15 Pleasant Street Connector, P.O. Box 9322, Framingham, MA, 01701-9322, US

(21) Appl. No.: 09/928,213

(22) Filed: Aug. 10, 2001

Related U.S. Application Data
(63) Continuation of application No. PCT/US00/03655, filed on Feb. 10, 2000.
(60) Provisional application No. 60/120,002, filed on Feb. 11, 1999.
(60) Provisional application No. 60/161,845, filed on Oct. 27, 1999.
(60) Provisional application No. 60/162,170, filed on Oct. 28, 1999.

(51) Int. Cl. A61K 48/00; C07H 21/04; A61K 9/127

(52) U.S. Cl. 514/44; 424/93.21; 536/23.5; 424/450

(57) ABSTRACT

Recombinant polynucleotide that contains a plurality of first polynucleotides encoding an antigenic peptide are provided by this invention. The first polynucleotides are operatively linked to each other to enhance translation of the polynucleotides to the antigenic peptide and binding of the antigenic peptide to MHC molecules. In a further embodiment, the recombinant contains a plurality of a second polynucleotide encoding multiple copies of antigenic peptides having an amino acid sequence that is different from the peptides encoded by the first polynucleotides. The polynucleotides are useful as cancer vaccines or in adoptive immunotherapy. In these embodiments, the polynucleotides encode a antigenic peptide that will induce an immune response to a tumor or cancer. Alternatively, the polypeptides encodes antigens that induce T cell anergy for use in autoimmune disorders. Still further, the antigen is a pathogenic antigen to induce an immune response against a pathogen such a virus or bacterial pathogen.

### CROSS-REFERENCE TO RELATED APPLICATIONS

This application claims priority under 35 U.S.C. § 119(e) to U.S. Provisional Application Serial Nos. 60/120,002; 60/161,845; and 60/162,170, filed Feb. 11, 1999; Oct. 27, 1999; and Oct. 28, 1999, respectively. The contents of these applications are hereby incorporated by reference into the present disclosure.

### TECHNICAL FIELD

This invention is in the field of immunotherapy and in particular, cancer vaccines.

### BACKGROUND OF THE INVENTION

The concept of creating vaccines to treat cancer has a long history. Early attempts at cancer vaccines used extracts of tumor tissue injected along with traditional adjuvants such as bacterial cell wall materials, to induce anti-tumor immune responses. The lack of success of such initial attempts at cancer vaccine therapy caused doubts as to whether any vaccine against cancer was possible. Nevertheless, efforts to perfect such methods continued and recent developments in the science of immunology and vaccine technology as well as advances in the understanding of cancer biology have stimulated renewed interest in this therapeutic approach. Recently, various technical approaches for inducing anti-cancer immunity have now shown promise, re-enforcing the potential of the concept.

Advances in the science of immunology have had a major impact on vaccine development. Key processes such as antigen presentation and recognition have been discovered and the importance of co-stimulatory molecules and various cell surface receptors in this process have been determined. The roles of different cell types in various parts of the process are now known and the contributions of such cells as antigen presenting leukocytes, B cells, helper T cells and cytotoxic T cells have been identified. In addition, the importance of stimulatory and inhibitory cytokines in modulating the process has been shown and the contributions of humoral, antibody based immunity, and cell-mediated immunity to resistance to various diseases has been studied.

Using contemporary molecular techniques, the complexity and subtlety of antigen processing and presentation has now begun to be appreciated. These advances have created a basis for development of novel vaccines with enhanced effectiveness.

At the same time that basic understanding of the molecular and cellular basis of immune responses has expanded, vaccine technology for the treatment of infectious diseases has also progressed. The introduction of techniques of molecular biology and genetic engineering has created novel methods for vaccination. Vaccine technology has moved beyond application of attenuated infectious agents to advanced methods such as recombinant subunit vaccines, administration of peptide molecules, creation of genetically engineered fusion proteins to enhance immunogenicity, design and delivery of recombinant multivalent virus vectors, and development of plasmid DNA vectors and other gene therapy based approaches.

Specific antigenic peptide epitopes have been identified and presented in various improved formats such as in the form of multimers covalently attached to a carrier protein or synthesized in bacteria as a fusion protein, or fusion proteins with B cell epitopes and helper T cell epitopes linked to stimulate production of antibodies to infectious agents. However, the goal of a cancer vaccine has not yet been achieved. Recent advances in cancer biology have shown that eliciting an immune response against neoplastic cells is challenging.

A variety of alternative mechanisms have been identified by which cancer cells may evade recognition and attack by the immune system. Pawelec G. et al. (1997) Crit. Rev. Oncog. 8:111-141; Hersey P. (1999) Pharmacol Ther. 81:111-119. Tumors may evade antigenic recognition due to their localization in a tissue that is naturally inaccessible to the immune system, such as the central nervous system, or the tumor may produce factors that block expression of cellular adhesion molecules on adjacent vascular endothelial cells preventing effective lymphocyte homing to the tumor. Weller and Fontana (1995) Brain Research News 21:128-151; Onrust S. V. et al. (1996) J. Clin. Invest. 97(1):54-64. In addition tumors may induce immunologic tolerance by large scale shedding of antigens into the serum and lymph or they may modulate antigen expression to avoid immune recognition and attack. Gopalkrishna P. (1998) Cell. Mol. Biol. 44:563-569.

Alternatively, even when appropriate tumor antigens are expressed and recognized, tumor cells may evade an immunologic response by various means. They can release immuno-suppressive cytokines like TGF-β, IL10 and VEGF that down regulate host immune responses Arteaga C. L. et al. (1993) J. Clin. Invest. 92:2569-2576; Chang H. L. et al. (1993) Cancer Research 53:4391-4398; Chouaib H. L. et al. (1997) Immunology Today 18:493-497; Gabrilovich D. et al. (1998) Blood 92:4150-4166. They can interfere with immune responses through increased production of prostaglandins or through the downregulation of MHC I molecules. Goodwin S. J. and Ceuppens J. L. (1985) In Prostaglandins and Immunity. Boston, Martinus Nijoff Publishing 1-34.; Cromme F. V. et al. (1994) J. of Exp. Med. 179:335-340. Tumor may present antigens but fail to produce essential co-stimulatory signals, such as the B7 cell surface molecule, necessary for T cell activation. Baskar S. et al. (1993) PNAS 90:5687-5690. Cancerous cells can acquire mutations that interfere with apoptotic pathways or actively attack activated cytotoxic T lymphocytes by activating apoptosis in these T cells. Weller M. et al. (1995) J. Clin. Inves. 95:2633-2643; Hug H. (1997) Biol. Chem. 378:1405-1412; Pitti R. M. et al. (1998) Nature 396:699-703. In addition the rapid growth kinetics of tumor cells may allow them to simply outgrow the capacity of the immune system to effectively keep their growth in check.

Thus, because of the complexity of the disease, an effective vaccine therapy will require a combination of methods to address the various complicating factors that have limited the effectiveness of attempts to date. Achieving optimal presentation of the cancer antigens is of central importance to achieving a strong response. This invention provides the methods and compositions to achieve this result.

### DESCRIPTION OF THE INVENTION

Provided herein is a recombinant polynucleotide that contains a plurality of first polynucleotides encoding an antigenic peptide. The first polynucleotides are operatively linked to each other to enhance translation of the polynucleotides to the antigenic peptide and binding of the antigenic peptide to MHC molecules. In a further embodiment, the recombinant polynucleotide contains a plurality of a second polynucleotide encoding multiple copies of antigenic peptides having an amino acid sequence that is different from the peptides encoded by the first polynucleotides.

The polynucleotides are useful as cancer vaccines or in adoptive immunotherapy. In these embodiments, the polynucleotides encode a antigenic peptide that will induce an immune response to a tumor or cancer. Alternatively, the polypeptides encodes antigens that induce T cell anergy for use in autoimmune disorders. Still further, the antigen is a pathogenic antigen to induce an immune response against a pathogen such a virus or bacterial pathogen.

An additional embodiment provides the recombinant polynucleotide described above which further contains a polynucleotide encoding alanine inserted between the plurality of polynucleotides endcoding the antigenic peptides. In a further embodiment, the recombinant polynucleotide contains a sequence that encodes an mRNA stability element or a viral internal ribosome binding site.

Gene delivery vehicles as well as host cells comprising the recombinant polynucleotides are further provided herein. In one aspect, the host cell is a dendritic cell such as an antigen presenting cell (APC) that processes and presents multiple copies of the epitope on the surface of the APC. In a further aspect, immune effector cells educated in the presence of the APC described herein are provided. The APC and immune effector cells are useful in methods of modulating an immune response.

### BRIEF DESCRIPTION OF THE FIGURES

FIG. 1 schematically shows one embodiment for linking the plurality of antigenic peptides.

FIGS. 2A through 2C are flow charts schematically showing the amplification steps that may be used to construct a recombinant polynucleotide of this invention.

FIG. 3 shows two separate embodiments of this invention. FIG. 3A shows the polynucleotides encoding gp209 antigenic peptide which are separated by a polynucleotide encoding 3 alanines. FIG. 3B is the sequence of the 3′UTR of an α- globin gene that may be inserted into the construct to enhance stability of the transcribed mRNA.

FIG. 4 is the sequence of a 9 copy recombinant polynucleotide.

FIGS. 5A and 5B are graphs that show that cells infected with the recombinant polynucleotides of this invention are more effective presenters of antigen to CTL as measured by the CTL assay. In FIG. 2B, MDA 231 cells transfected with vectors comprising a plurality of polypeptides encoding the antigenic peptide gp100 209 enhances cell lysis as assayed by CTL. An incremental increase in the percent lysis was observed in proportion to the number of copies of the epitopes.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, microbiology, cell biology and recombinant DNA. These methods are described in the following publications. See, e.g., Sambrook, et al. MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.); “PCR: A PRACTICAL APPROACH” (M. MacPherson, et al., IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane, eds. (1988)); and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

A “gene” refers to a polynucleotide containing at least one open reading frame that is capable of encoding a particular polypeptide or protein after being transcribed and translated.

A “gene product” refers to the amino acid (e.g., peptide or polypeptide) generated when a gene is transcribed and translated.

The term “peptide” is used in its broadest sense to refer to a compound of two or more subunit amino acids, amino acid analogs, or peptidomimetics. The subunits may be linked by peptide bonds. In another embodiment, other bonds may link the subunit, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “cDNAs” refers to complementary DNA, that is mRNA molecules present in a cell or organism made in to cDNA with an enzyme such as reverse transcriptase. A “cDNA library” is a collection of all of the mRNA molecules present in a cell or organism, all turned into cDNA molecules with the enzyme reverse transcriptase, then inserted into “vectors”.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′ —OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “promoter” is a region on a DNA molecule to which an RNA polymerase binds and initiates transcription. In an operon, the promoter is usually located at the operator end, adjacent but external to the operator. The nucleotide sequence of the promoter determines both the nature of the enzyme that attaches to it and the rate of RNA synthesis.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

A “composition” is intended to mean a combination of active agent and another compound or composition, inert (for example, a detectable agent or label or a pharmaceutically acceptable carrier) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton 1975)).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

A “gene delivery vehicle” is defined as any molecule that can carry inserted polynucleotides into a host cell. Examples of gene delivery vehicles are liposomes, cationic liposomes, viruses, such as baculovirus, adenovirus, adeno-associated virus, and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and the inserted polynucleotide. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form, which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a polynucleotide to be inserted. Adenoviruses (Ads) are a relatively well characterized, homogenous group of viruses, including over 50 serotypes. (see, e.g., WO 95/27071). Ads are easy to grow and do not require integration into the host cell genome. Recombinant Ad-derived vectors, particularly those that reduce the potential for recombination and generation of wild-type virus, have also been constructed. (see, WO 95/00655; WO 95/11984). Wild-type AAV has high infectivity and specificity integrating into the host cells genome. (Hermonat and Muzyczka (1984) PNAS USA 81:6466-6470; Lebkowski, et al. (1988) Mol. Cell. Biol. 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; stabilizing elements 3′ to the inserted polynucleotide, and T7 and SP6 RNA promoters for in vitro transcription of sense and antisense RNA. Other means are well known and available in the art.

“Host cell” is intended to include any individual cell or cell culture which can be or have been recipients for vectors or the incorporation of exogenous polynucleotides, polypeptides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, plant cells, insect cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antigen” as used herein means a substance that causes an immune system response. An “antigenic peptide” is the minimal fragment of the antigen that stimulates the production of the immune response.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

A synthetic peptide of the invention is said to “correspond” to a native epitope if the peptide binds to the same TCR as the natural epitope. In some embodiments, a peptide of the invention increases or decreases an immune response specific to the native epitope.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

An “mRNA stability element” is intended to include those sequences and factors that interact to increase the stability or half life of the mRNA.

The terms “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an a chain encoded in the MHC associated noncovalently with B2-microglobulin. Class I MHC molecules are expressed by nearly all nucleated cells and have been shown to function in antigen presentation to CD8+ T cells. Class I molecules include HLA-A, -B, and -C in humans. Class I molecules generally bind peptides 8-10 amino acids in length. Class II MHC molecules also include membrane heterodimeric proteins consisting of noncovalently associated α and β chains. Class II MHC are known to participate in antigen presentation to CD4+ T cells and, in humans, include HLA-DP, -DQ, and DR. Class II molecules generally bind peptides 12-20 amino acid residues in length. The term “MHC restriction” refers to a characteristic of T cells that permits them to recognize antigen only after it is processed and the resulting antigenic peptides are displayed in association with either a self class I or class II MHC molecule. Methods of identifying and comparing MHC are well known in the art and are described in Allen et al. (1994) Human Imm. 40:25-32; Santamaria et al. (1993) Human Imm. 37:39-50 and Hurley et al. (1997) Tissue Antigens 50:401-415.

The term “antigen-presenting matrix”, as used herein, intends a molecule or molecules which can present antigen in such a way that the antigen can be bound by a T-cell antigen receptor on the surface of a T cell. An antigen-presenting matrix can be on the surface of an antigen-presenting cell (APC), on a vesicle preparation of an APC, or can be in the form of a synthetic matrix on a solid support such as a bead or a plate. An example of a synthetic antigen-presenting matrix is purified MHC class I molecules complexed to β2-microglobulin, or purified MHC Class II molecules, or functional portions thereof, attached to a solid support.

The term “antigen presenting cell”, as used herein, intends any cell which presents on its surface an antigen in association with a major histocompatibility complex molecule, or portion thereof, or, alternatively, one or more non-classical MHC molecules, or a portion thereof. Examples of suitable APCs are discussed in detail below and include, but are not limited to, whole cells such as macrophages, dendritic cells, B cells, hybrid APCs, and foster antigen presenting cells. Methods of making hybrid APCs have been described. See, for example, International Patent Application No. WO 98/46785; and WO 95/16775.

Dendritic cells (DCs) are potent antigen-presenting cells. It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called co-stimulatory signals, is neither antigen-specific nor MHC-restricted, and can lead to a full proliferation response of T cells and induction of T cell effector functions in the presence of the first type of signals. As used herein, “dendritic cell” is to include, but not be limited to a pulsed dendritic cell, a foster cell or a dendritic cell hybrid.

The term “modulate an immune response” includes inducing (increasing, eliciting) an immune response; and reducing (suppressing) an immune response. An immunomodulatory method (or protocol) is one that modulates an immune response in a subject.

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule, which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹Cr-release assays, or ³H-thymidine uptake assays.

The term “immune effector cells” refers to cells capable of binding an antigen or which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates. Certain diseased tissue expresses specific antigens and CTLs specific for these antigens have been identified. For example, approximately 80% of melanomas express the antigen known as gp100.

The term “immune effector molecule”, as used herein, refers to molecules capable of antigen-specific binding, and includes antibodies, T cell antigen receptors, and MHC Class I and Class II molecules.

A “naive” immune effector cell is an immune effector cell that has never been exposed to an antigen.

As used herein, the term “educated, antigen-specific immune effector cell”, is an immune effector cell as defined above, which has encountered antigen and which is specific for that antigen. An educated, antigen-specific immune effector cell may be activated upon binding antigen. “Activated” implies that the cell is no longer in G₀ phase, and begins to produce cytokines characteristic of the cell type. For example, activated CD4+ T cells secrete IL-2 and have a higher number of high affinity IL-2 receptors on their cell surfaces relative to resting CD4+ T cells.

Specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called co-stimulatory signals, is neither antigen-specific nor MHC-restricted, and can lead to a full proliferation response of T cells and induction of T cell effector functions in the presence of the first type of signals. As used herein, “dendritic cell” is to include, but not be limited to a pulsed dendritic cell, a foster cell or a dendritic cell hybrid.

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz R. H. (1990) Science 248:1349-1356; Jenkins M. K. (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so-called “professional” APCs. Several molecules have been shown to enhance co-stimulatory activity. These are heat stable antigen (HSA) (Liu Y. et al. (1992) J. Exp. Med. 175:437-445), chondroitin sulfate-modified MHC invariant chain (Ii-CS) (Naujokas M. F. et al. (1993) Cell 74:257-268), intracellular adhesion molecule 1 (ICAM-1) (Van Seventer G. A. (1990) J. Immunol. 144:4579-4586), B7-1, and B7-2/B70 (Schwartz R. H. (1992) Cell 71:1065-1068). These molecules each appear to assist co-stimulation by interacting with their cognate ligands on the T cells. Co-stimulatory molecules mediate co-stimulatory signal(s) which are necessary, under normal physiological conditions, to achieve full activation of naive T cells. One exemplary receptor-ligand pair is the B7 co-stimulatory molecule on the surface of APCs and its counter-receptor CD28 or CTLA-4 on T cells (Freeman et al. (1993) Science 262:909-911; Young et al. (1992) J. Clin. Invest. 90: 229; Nabavi et al. (1992) Nature 360:266-268). Other important co-stimulatory molecules are CD40, CD54, CD80, CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter. It is intended, although not always explicitly stated, that molecules having similar biological activity as wild type or purified co-stimulatory molecules (e.g., recombinantly produced or muteins thereof) are intended to be used within the spirit and scope of the invention.

A peptide or polypeptide of the invention may be preferentially recognized by antigen-specific immune effector cells, such as B cells and T cells. In the context of T cells, the term “recognized” intends that a peptide or polypeptide of the invention, comprising one or more synthetic antigenic epitopes, is recognized, i.e., is presented on the surface of an APC together with (i.e., bound to) an MHC molecule in such a way that a T cell antigen receptor (TCR) on the surface of an antigen-specific T cell binds to the epitope wherein such binding results in activation of the T cell. The term “preferentially recognized” intends that a polypeptide of the invention is substantially not recognized, as defined above, by a T cell specific for an unrelated antigen. Assays for determining whether an epitope is recognized by an antigen-specific T cell are known in the art and are described herein.

The term “autogeneic” or “autologous”, as used herein, indicates the origin of a cell. Thus, a cell being administered to an individual (the “recipient”) is autogeneic if the cell was derived from that individual (the “donor”) or a genetically identical individual. An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic” as used herein, indicates the origin of a cell. Thus, a cell being administered to individual (the “recipient”) is allogeneic if the cell was derived from an individual not genetically identical to the recipient; in particular, the term relates to non-identity in expressed MHC molecules. An allogeneic cell can also be a progeny of an allogeneic cell. The term also indicates that cells of different cell types are derived from genetically non-identical donors, or if they are progeny of cells derived from genetically non-identical donors. For example, an APC is said to be allogeneic to an effector cell if they are derived from genetically non-identical donors.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., deregulated cell division). Neoplastic cells can be malignant or benign.

“Suppressing” tumor growth indicates a growth state that is curtailed when compared to growth without contact with educated, antigen-specific immune effector cells described herein. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1I), interleukin-11 (IL-11), MIP-1I, leukemia inhibitory factor (LIF), c-kit ligand, thrombopoietin (TPO) and flt3 ligand. The present invention also includes culture conditions in which one or more cytokine is specifically excluded from the medium. Cytokines are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). It is intended, although not always explicitly stated, that molecules having similar biological activity as wild type or purified cytokines (e.g., recombinantly produced or muteins thereof) are intended to be used within the spirit and scope of the invention.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein, “solid phase support” is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel™, Rapp Polymere, Tubingen, Germany) or polydimethylacrylamide resin (obtained from Milligen/Biosearch, California). In a preferred embodiment for peptide synthesis, solid phase support refers to polydimethylacrylamide resin.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

An “enriched” population of cells, as used herein, means that a cell population is at least about 50-fold, more preferably at least about 500-fold, and even more preferably at least about 5000-fold or more enriched from an original naive cell population. The proportion of the enriched cell population, which comprises antigen-specific cells, can vary substantially, from less than 10% up to 100% antigen-specific cells.

Embodiments of the Invention

Degradation of intracellular proteins within an antigen presenting cell leads to the generation of a myriad of peptides that compete for binding to and presentation by a finite number of major histocompatibility (MHC) protein complexes. The likelihood that one peptide sequence will be presented in the surface of an APC will be dependent upon the relative abundance of that particular peptide within the APC as well as the affinity of the peptide for available MHC molecules. High levels of peptide presentation by APCs is likely to favor the promotion, amplification and maintenance of an immune response directed towards that particular peptide sequence. However competition between difference antigenic peptides for binding to MHC molecules will thwart the attainment of high level presentation of any one specific peptide sequence on an APC.

Multiple copies of a polynucleotide encoding a specific antigenic peptide sequence are linked together and placed under the control of a strong promoter such that transcription and subsequent translation of the polynucleotide leads to the formation of a polypeptide consisting of multiple copies of the desired specific peptide sequence. Intracellular processing of the polypeptide will liberate the individual copies of the antigenic peptide, each of which are now available to compete with other intracellular peptides for binding to an MHC molecule. Thus unlike a naturally occurring protein where an antigenic peptide sequence will appear once and proteolysis of that protein will give rise to a single copy of the antigenic peptide, proteolysis of the engineered polypeptide of the present invention gives rise to multiple copies of the antigenic peptide and improves the likelihood that the specific peptide will be presented by an APC.

Polynucleotides

This invention provides a recombinant polynucleotide containing a plurality of a first polynucleotide encoding an identical antigenic peptide. As used herein, a plurality shall mean at least two and more preferably, three or more copies of the same polynucleotide. In the embodiments, separate plurality includes at least 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 30, or 35, or 40 or 45 or more than 50 copies of the identical antigenic peptide. The plurality of polynucleotides will only be limited by the capacity of the vector. A plurality, as defined above, or more different polynucleotides (referred to herein as “second polynucleotides”) can be combined in the same recombinant polynucleotide. The polynucleotides are arranged 5′ to 3′ in a head to tail fashion or concatamer. The first polynucleotides also are arranged and combined such that they are operatively linked to each other to enhance translation of the polynucleotides to the antigenic peptides and binding of the antigenic peptides to MHC molecules. In one embodiment, a polynucleotide encoding a methionine residue required for translational initiation is appended to the 5′end of the concatamer and the entire sequence is placed under the control of a strong transcriptional promoter (e.g., a CMV promoter) within a viral vector. In another aspect, the recombinant polynucleotide encodes a mRNA stability element appended 3′ to the polynucleotides encoding the antigen. Examples, of mRNA stability elements include, but are not limited to the murine or human 3′ UTR of the α-globulin gene (see, Wang and Liebhaber (1996) EMBO J. 15(18):5040-5051 and Holick and Liebhaber, (1997) PNAS USA 94:2410-2414, respectively) and functionally equivalent polynucleotides thereof. These include polynucleotides that hybridize to the 3′ UTR of the murine and human α-globulin genes under conditions of moderate or high stringency, as well as polynucleotides having the same biological activity and that are at least 80%, 90% or 95% homologous thereto as determined by a sequence alignment program under default parameters. Alternative examples of 3′ UTR mRNA stability elements include but are not limited to the GLUT1 3′ UTR (McGowan et al. (1997) J. Biol. Chem. 272(2):1331-1337) and the 3′ UTR of the tau mRNA (Aronov et al. (1999) J. Mol. Neurosci. 12(2):131-145).

In yet another aspect, the recombinant polynucleotides comprise sequences that code for at least three amino acids, comprising alanine or other amino acids with hydrocarbon side chains such as glycine, valine, leucine, and isoleucine. These amino acids are inserted between the polynucleotides encoding the antigenic peptides to facilitate processing and presentation of the antigenic peptides.

In yet a further aspect, the recombinant polynucleotide comprises a viral internal ribosome entry site or other enhancer element to facilitate expresssion of the polynucleotides encoding the epitopes.

In another aspect, the recombinant polynucleotide further comprises a third polynucleotide encoding a cytokine and/or a costimulatory molecule. Examples of cytokines and costimulatory molecules are provided in the definition section, above.

The polynucleotides used in this invention encode native, natural or wild type antigenic peptides as well as synthetic antigenic peptides. The antigen can be “self” or foreign, and can be derived from any organism. The antigen may be autologous or heterologous (i.e., allogeneic or a homolog from a isolated species, e.g., a murine antigen administered to a human patient.) Examples include, but are not limited to previously characterized tumor-associated antigens such as gp 100 (Kawakami et al.(1997) Intern. Rev. Immunol. 14:173-192), MUC-1 (Henderson et al. (1996) Cancer Res. 56:3762-3770), MART-1 (Kawakami et al. (1994) Proc. Natl. Acad. Sci. 91:3515-3519; Kawakami et al. (1997) Intern. Rev. Immunol. 14:173-192; Ribas et al. (1997) Cancer Res. 57:2865-2869), HER-2/neu (U.S. Pat. No. 5,520,214), MAGE (PCT/US92/04354) HPV16, 18E6 and E7 (Ressing et al. (1996) Cancer Res. 56(1):582-588; Restifo (1996) Current Opinion in Immunol. 8:658-663; Stern (1996) Adv. Cancer Res. 69:175-211; Tindle et al. (1995) Clin. Exp. Immunol.101:265-271; van Driel et al. (1996) Annals of Medicine 28:471-477) CEA (U.S. Pat. No. 5,274,087) and 4,898,814; Brichard et al. (1993) J. Exp. Med. 178:489-49); tyrosinase related proteins 1 or 2 (TRP-1 and TRP-2), NY-ESO-1 (Chen et al. (1997) Proc. Natl. Acad. Sci. U.S.A. 94:1914-18), or the GA733 antigen (U.S. Pat. No. 5,185,254). Synthetic antigenic peptide epitopes of the present invention can be designed based on known amino acid sequences of antigenic peptide epitopes.

Peptide epitopes associated with pathogenic organisms include peptides from the influenza nucleoprotein composed of residues 365-80 (NP365-80), NP50-63, and NP147-58 and peptides from influenza hemagglutinin HA202-21 and HA523-45, defined previously in class I restricted cytotoxicity assays. Perkins et al. (1989) J. Exp. Med. 170: 279-289. Enhanced efficiency of association of such polypeptides to specific class I molecules on antigen presenting cells in vivo has major implications for the use of these synthetic peptides as influenza vaccines. Other examples of synthetic peptides containing known epitopes that can be recognized by MHC-restricted CTLs include influenza strain A/Jap/57 hemagglutinin protein, residues 508-530; influenza strain A/PR8/34 nucleoprotein residues 360-385; HIV Pol (reverse transcriptase) residues 203-219; Sendai virus nucleoprotein peptide, residues 324-332; and the vesicular stomatitis nucleotide protein, amino acid residues 52-59. Peptides representing epitopes displayed by the malarial parasite Plasmodium falciparum have been described. U.S. Pat. No. 5,609,872.

An example of a self-tissue antigen recognized in autoimmune disorders is the acetylcholine receptor (AChR) which is recognized in myasthenia gravis. The T lymphocyte response in these patients may be directed to additional epitopes on the AChR. Although the majority of T cell recognition sites are on the subunit, T cells also recognize epitopes in the other subunits. Indeed, T cells from patients have been shown to respond to more than 30 different AChR-derived peptides. Examples of AChR epitopes are the following:

HM1: Y N L K W N Y N L K W N Y N L K W N

HM2: P D D Y G G P D D Y G G P D D Y G G

HM3: V K K I H I V K K I H I V K K I H I

HM4: K W N P D D K W N P D D K W N P D D Y

HM5: Y G G V K K Y G G V K K Y G G V K K

HM6: W N P D D Y G G V K W N P D D Y G G V K

Another class of self-antigens for which antigenic epitopes have been described is human chorionic gonadotropin (hCG) beta subunit. U.S. Pat. No. 5,733,553. These epitopes find utility in contraceptive methods.

This list of peptides is exemplary only and is not intended to limit the Class I or Class II peptides that can be modified for use in the methods of the present invention can be employed. Class I and Class II peptides that can be used with the present invention can also be determined empirically in accordance with techniques known in the art. For example, the peptides that are displayed by a variety of different class I molecules can be defined for a given pathogen-related antigen by infecting somatic cells of given class I HLA types with the pathogen of interest. The peptides that bind to the class I molecules after normal intracellular processing are then eluted from the target cell surface and subjected to sequence analysis in accordance with known techniques. Alternatively, overlapping peptides from a given pathogen-related protein can be synthesized and analyzed for their ability to bind to the various Class I and Class II HLA types. Alternatively, a method such as SPHERE, which is described in more detail below, can be used to identify antigenic epitopes.

The invention also encompasses polynucleotides which differ from that of the polynucleotides described above, but which produce the same phenotypic effect, such as the allele, splice variant and homolog. These altered, but phenotypically equivalent polynucleotides are referred to “equivalent nucleic acids.” This invention also encompasses polynucleotides characterized by changes in non-coding regions that do not alter the phenotype of the polypeptide produced therefrom when compared to the polynucleotide herein. This invention further encompasses polynucleotides, which hybridize to the polynucleotides of the subject invention under conditions of moderate or high stringency.

Alternatively, biologically equivalent polynucleotides can be identified using sequence homology searches. Several embodiments of biologically equivalent polynucleotides are within the scope of this invention, e.g., those characterized by possessing at least 75%, or at least 80%, or at least 90% or at least 95% sequence homology as determined using a sequence alignment program under default parameters correcting for ambiguities in the sequence data, changes in nucleotide sequence that do not alter the amino acid sequence because of degeneracy of the genetic code, conservative amino acid substitutions and corresponding changes in nucleotide sequence, and variations in the lengths of the aligned sequences due to splicing variants or small deletions or insertions between sequences that do not affect function.

A variety of software programs are available in the art. Non-limiting examples of these programs are BLAST family programs including blastn, blastp, blastx, tblastn, and tblastx (BLAST is available from the worldwide web at http://www.ncbi.nlm.nih.gov/BLAST/), FastA, Compare, DotPlot, BestFit, GAP, FrameAlign, ClustalW, and PileUp. These programs can be obtained commercially in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at http://www.sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS. Sequence similarity can be discerned by aligning the polynucleotide sequence of interest or a fragment thereof against a DNA sequence database. Alternatively, the polynucleotide sequence can be translated into six reading frames; the predicted peptide sequences of all possible reading frames are then compared to individual sequences stored in a protein database such as s done using the BLASTX program.

Parameters for determining the extent of homology set forth by one or more of the aforementioned alignment programs are well established in the art. They include but are not limited to p value, percent sequence identity and the percent sequence similarity. P value is the probability that the alignment is produced by chance. For a single alignment, the p value can be calculated according to Karlin et al. (1990) PNAS 87: 2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical. A putative equivalent sequence is considered to lack substantial homology with a known sequence when the regions of alignment of comparable length exhibit less than 30% of sequence identity, more preferably less than 20% identity, even more preferably less than 10% identity.

The polynucleotides of the invention can comprise additional sequences, such as additional coding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

The polynucleotides of this invention can be isolated using methods known in the art and described in the literature, e.g., replicated using PCR. The PCR technology is the subject matter of U.S. Pat. Nos. 4,683,195, 4,800,159, 4,754,065, and 4,683,202 and described in PCR: The Polymerase Chain Reaction (Mullis et al. eds, Birkhauser Press, Boston (1994)) or MacPherson, et al. (1991) and (1994), supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook, et al. (1989) supra. For instance, mRNA can be isolated using various lytic enzymes or chemical solutions according to the procedures set forth in Sambrook, et al. (1989), supra or extracted by nucleic-acid-binding resins following the accompanying instructions provided by manufactures.

A preferred amplification method is PCR. PCR conditions used for each reaction are empirically determined. A number of parameters influence the success of a reaction. Among them are annealing temperature and time, extension time, Mg2+ ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are CMV, SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)), which contains maps, functional properties, commercial suppliers and a reference to GenEMBL accession numbers for various suitable vectors. Preferable, these vectors are capable of transcribing RNA in vitro or in vivo.

Expression vectors containing these nucleic acids are useful to obtain host vector systems to produce proteins and polypeptides as well as in gene therapy applications. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook, et al. (1989) supra. Procaryotic cell systems are useful to assay expression efficacy of various combinations of antigenic peptides combinations. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook, et al. (1989) supra for this methodology. Thus, this invention also provides a host cell, e.g. a mammalian cell, an animal cell (rat or mouse), a human cell, or a procaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller A. D. et al. (1989) BioTechniques 7:980-990). The methodology of using replication-incompetent retroviruses for retroviral-mediated gene transfer of gene markers is well established (Correll et al. (1989) PNAS USA 86:8912; Bordignon (1989) PNAS USA 86:8912-52; Culver K. (1991) PNAS USA 88:3155; and Rill D. R. (1991) Blood 79(10):2694-700. Clinical investigations have shown that there are few or no adverse effects associated with the viral vectors, see Anderson (1992) Science 256:808-13.

Methods for Designing Synthetic Antigenic Peptide Epitopes

Synthetic antigenic peptide epitopes can be designed based on natural peptide epitopes identified using any method known in the art. The following provides non-limiting examples of methods that can be used. In addition, modifications or combinations of any of the following methods can be used. For example, modifications of the SAGE and the SPHERE methods are described in International Patent Application No. PCT/US99/01462.

Methods involving isolating and assaying MHC molecules from antigen presenting cells can be used to identify peptides bound to the MHC molecules. Chicz and Urban (1994) Immunol. Today 1-5:155-160. Bacteriophage “phage display” libraries can also be constructed. Using the “phage method” (Scott and Smith (1990) Science 249:386-390; Cwirla et at. (1990) PNAS USA. 87:6378-6382; Devlin et al. (1990) Science 249:404-406), very large libraries can be constructed (10⁶-10⁸ chemical entities). Other methods to identify peptide epitopes which can be used involve primarily chemical methods, of which the Geysen method (Geysen et al. (1986) Molecular Immunology 23:709-715; Geysen et al. (1987) J. Immunologic Method 102:259-274) and the method of Fodor et al. (1991) Science 251:767-773) are examples. Furka et al. (1988) 14th International Congress of Biochemistry, Volume 5. Abstract FR:013; Furka, (1991) Int. J. Peptide Protein Res. 37:487-493). Houghton (U.S. Pat. No. 4,631,211 issued December 1986) and Rutter et al. (U.S. Pat. No. 5,101,175, issued Apr. 23, 1991) describe methods to produce a mixture of peptides that can be tested as agonists or antagonists. Other methods which can be employed involve use of synthetic libraries (Needels et al. (1993) Proc. Natl. Acad. Sci. USA 90:10700-4; Ohlmeyer et al. (1993) Proc. Natl. Acad. Sci. USA 90:10922-10926; Lam et al., International Patent Publication No. WO 92/00252, each of which is incorporated herein by reference in its entirety), and the like can be used to screen for receptor ligands. Techniques based on cDNA subtraction or differential display have been described amply in the literature and can also be used. see, for example, Hedrick et al. (1984) Nature 308:149; and Lian and Pardee (1992) Science 257:967. The expressed sequence tag (EST) approach is a valuable tool for gene discovery (Adams et al. (1991) Science 252:1651), as are Northern blotting, RNase protection, and reverse transcriptase-polymerase chain reaction (RT-PCR) analysis (Alwine et al. (1977)PNAS USA 74:5350; Zinn et al. (1983) Cell 34:865; Veres et al. (1987) Science 237:415). Another technique which can be used is the “pepscan” technique (Van der Zee (1989) Eur. J. Immunol. 19:43-47) in which several dozens of peptides are simultaneously synthesized on polyethylene rods arrayed in a 96-well microliter plate pattern, similar to an indexed library in that the position of each pin defines the synthesis history on it. Peptides are then chemically cleaved from the solid support and supplied to irradiated syngeneic thymocytes for antigen presentation. A cloned CTL line is then tested for reactivity in a proliferation assay monitored by ³H-thymidine incorporation.

Another method which can be used is the SAGE technique, which allows a rapid, detailed analysis of thousands of transcripts. The SAGE method is described in U.S. Pat. No. 5,695,937. SAGE is based on two principles. First, a short nucleotide sequence tag (9 to 10 bp) contains sufficient information content to uniquely identify a transcript provided it is isolated from a defined position within the transcript. For example, a sequence as short as 9 bp can distinguish 262,144 transcripts (Fields et al. (1994) Nature Genet. 7:345) given a random nucleotide distribution at the tag site, whereas current estimates suggest that even the human genome only encodes about 80,000 transcripts. Fields et al. (1994) Nature Genet. 7:345. Second, concatenation of short sequence tags allows the efficient analysis of transcripts in a serial manner by sequencing of multiple tags within a single clone. As with serial communication by computers, wherein information is transmitted as a continuous string of data, serial analysis of the sequence tags requires a means to establish the register and boundaries of each tag.

An alternative method to identify antigenic peptides is disclosed in WO99/37797 (published Jul. 29, 1999). Briefly, the invention provides a method for identifying epitopes and antigens recognized by immune effector cells and the polynucleotides that encode them. In one embodiment, the methods combine identifying the polynucleotides that encode sequence motifs of such antigens and identification of polynucleotides which are aberrantly expressed in the cells recognized by the immune effector cells. By comparison of these polynucleotide sequences, novel antigens that are recognized by immune effector cells can be identified. This invention also provides a method for identifying and cloning genes that encode the antigens as identified herein as well as methods of using genes and the proteins or polypeptides encoded by the genes.

The SPHERE approach (WO 97/35035) utilizes combinatorial peptide libraries synthesized on polystyrene beads wherein each bead contains a pure population of a unique peptide that can be chemically released from the beads in discrete aliquots. Released peptide from pooled bead arrays are screened using methods to detect T cell activation, including, for example, ³H-thymidine incorporation (for CD4+ or CD8+ T cells), ⁵¹Cr-release assay (for CTLs) or IL-2 production (for CD4+ T cells) to identify peptide pools capable of activating a T cell of interest. By utilizing an iterative peptide pool/releasing strategy, it is possible to screen more than 10⁷ peptides in just a few days. Analysis of residual peptide on the corresponding positive beads (>100 pmoles) allows rapid and unambiguous identification of the epitope sequence.

A brief overview of an assay to identify peptides binding to CTLs is as follows: roughly speaking, ten 96-well plates with 1000 beads per well will accommodate 10⁶ beads; ten 96-well plates with 100 beads per well will accommodate 10⁵ beads. In order to minimize both the number of CTL cells required per screen and the amount of manual manipulations, the eluted peptides can be further pooled to yield wells with any desired complexity. For example, based on experiments with soluble libraries, it is possible to screen 10⁷ peptides in 96-well plates (10,000 peptides per well) with as few as 2×10⁶ CTL cells. After cleaving a percentage of the peptides from the beads, incubating them with gamma-irradiated foster APCs and the cloned CTL line(s), positive wells detemined by ³H-thymidine incorporation are further examined. Alternatively, as pointed out above, cytokine production or cytolytic ⁵¹Cr-release assays may be used. Coulie et al. (1992) Int. J. Cancer 50:289-291. Beads from each positive well will be separated and assayed individually as before, utilizing an additional percentage of the peptide from each bead. Positive individual beads will then be decoded, identifying the reactive amino acid sequence. Analysis of all positives will give a partial profile of conservatively substituted epitopes which stimulate the CTL clone tested. At this point, the peptide can be resynthesized and retested. Also, a second library (of minimal complexity) can be synthesized with representations of all conservative substitutions in order to enumerate the complete spectrum of derivatives tolerated by a particular CTL. By screening multiple CTLs (of the same MHC restriction) simultaneously, the search for crossreacting epitopes is greatly facilitated.

The described method for the identification of CD8⁺ MHC Class I-restricted CTL epitopes can be applied to the identification of CD4⁺ MHC Class II-restricted CD4⁺ T cell eptitopes. In this case, MHC Class II allele-specific libraries are synthesized such that haplotype-specific anchor residues are represented at the appropriate positions. MHC Class II agretopic motifs have been identified for the common alleles. Rammensee (1995) Curr. Opin. Immunol. 7:85-96; Altuvia et al. (1994) Mol. Immunol. 24:375-379; Reay et al. (1994) J. Immunol. 152:3946-3957; Verreck et al. (1994) Eur. J. Immunol. 24:375-379; Sinigaglia and Hammer (1994) Curr. Opin. Immunol. 6:52-56; Rotzschke and Falk (1994) Curr. Opin. Immunol. 6:45-51. The overall length of the peptides will be 12-20 amino acid residues, and previously described methods may be employed to limit library complexity. The screening process is identical to that described for MHC Class I-associated epitopes except that the antigen presenting matrix would comprise MHC Class II molecules and any required co-stimulatory molecules. MHC Class II molecule-bearing antigen-presenting cells include, but are not limited to, B lymphoblastoid cell lines (B-LCL). As one example, previously characterized B-LCLs that are defective in antigen processing, thus allowing specific presentation of exogenously added antigen, can be employed. Mellins et al. (1991) J. Exp. Med. 174:1607-1615. The libraries are screened for reactivity with isolated CD4⁺ MHC Class II allele-specific CD4+ cells. Reactivity may be measured by ³H-thymidine incorporation according to the method of Mellins et al., supra, or by any of the methods previously described for MHC Class I-associated epitope screening.

Host Cells Comprising Recombinant Polynucleotides of the Invention

The invention further provides isolated host cells comprising the recombinant polynucleotides described above and host cell comprising the polypeptides encoded by the polynucleotides. Host cells, include eucaryotic and procaryotic cells, e.g., insect, mammalian, simian, murine, bacterial, or yeast cells. When the host cell is a dendritic cell such as an APC, the host cells present more than one copy of peptide or peptides on the surface of the cells. Isolated host cells which present the polypeptides of this invention in the context of MHC molecules are further useful to induce an immune response in a subject as well as to expand and isolate a population of educated, antigen-specific immune effector cells. The immune effector cells, e.g., cytotoxic T lymphocytes, are produced by culturing naive immune effector cells with antigen-presenting cells cells which present the polypeptides in the context of MHC molecules on the surface of the APCs. The population can be purified using methods known in the art, e.g., FACS analysis or FICOLL™ gradient. The methods to generate and culture the immune effector cells as well as the populations produced thereby also are the inventor's contribution and invention. Pharmaceutical compositions comprising the cells and pharmaceutically acceptable carriers are useful in adoptive immunotherapy. Prior to administration in vivo, the immune effector cells are screened in vitro for their ability to lyse or react with the cell of interest.

In some of these embodiments, isolated host cells are APCs. APCs include, but are not limited to, dendritic cells (DCs), monocytes/macrophages, B lymphocytes or other cell type(s) expressing the necessary MHC/co-stimulatory molecules.

In some embodiments, the immune effector cells and/or the APCs are genetically modified. Using standard gene transfer, genes coding for co-stimulatory molecules and/or stimulatory cytokines can be inserted prior to, concurrent to or subsequent to expansion of the immune effector cells.

The APCs are generally alive but can also be irradiated, mitomycin C treated, attenuated, or chemically fixed. Further, the APCs need not be whole cells. Instead, vesicle preparations of APCs can be used.

APCs can be genetically modified, i.e., transfected with a recombinant polynucleotide construct such that they express a polypeptide or an RNA molecule which they would not normally express or would normally express at lower levels. Examples of polynucleotides include, but are not limited to, those which encode an MHC molecule; a co-stimulatory molecule such as B7; and a peptide or polypeptide of the invention.

Cells which do not normally function in vivo in mammals as APCs can be modified in such a way that they function as APCs. A wide variety of cells can function as APCs when appropriately modified. Examples of such cells are insect cells, for example Drosophila or Spodoptera; and foster cells, such as the human cell line T2 commercially available from the American Type Culture Collection (ATCC) under accession No. CRL-1992). For example, expression vectors which direct the synthesis of one or more antigen-presenting polypeptides, such as MHC molecules, optionally also accessory molecules such as B7, can be introduced into these cells to effect the expression on the surface of these cells antigen presentation molecules and, optionally, accessory molecules or functional portions thereof. Alternatively, antigen-presenting polypeptides and accessory molecules which can insert themselves into the cell membrane can be used. For example, glycosylphosphotidylinositol (GPI)-modified polypeptides can insert themselves into the membranes of cells. Hirose et al. (1995) Methods Enzymol. 250:582-614; and Huang et al. (1994) Immunity 1:607-613. Accessory molecules include, but are not limited to, co-stimulatory antibodies such as antibodies specific for CD28 , CD80, or CD86; costimulatory molecules, including, but not limited to, B7.1 and B7.2; adhesion molecules such as ICAM-1 and LFA-3; and survival molecules such as Fas ligand and CD70. See, for example, PCT Publication No. WO 97/46256.

The following is a brief description of two fundamental approaches for the isolation of APC. These approaches involve (1) isolating bone marrow precursor cells (CD34⁺) from blood and stimulating them to differentiate into APC; or (2) collecting the precommitted APCs from peripheral blood. In the first approach, the patient must be treated with cytokines such as GM-CSF to boost the number of circulating CD34⁺ stem cells in the peripheral blood.

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/nonadherence steps (Freudenthal et al. (1990) PNAS 87:7698-7702); Percoll gradient separations (Mehta-Damani et al. (1994) J. Immunol. 153:996-1003); and fluorescence activated cell sorting techniques (Thomas et al. (1993) J. Immunol. 151:6840-52).

One technique for separating large numbers of cells from one another is known as countercurrent centrifugal elutriation (CCE). In this technique, cells are subject to simultaneous centrifugation and a washout stream of buffer which is constantly increasing in flow rate. The constantly increasing countercurrent flow of buffer leads to fractional cell separations that are largely based on cell size.

In one aspect of the invention, the APC are precommitted or mature dendritic cells which can be isolated from the white blood cell fraction of a mammal, such as a murine, simian or a human (See, e.g., WO 96/23060). The white blood cell fraction can be from the peripheral blood of the mammal. This method includes the following steps: (a) providing a white blood cell fraction obtained from a mammalian source by methods known in the art such as leukopheresis; (b) separating the white blood cell fraction of step (a) into four or more subfractions by countercurrent centrifugal elutriation, (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c), and (e) collecting the enriched fraction of step (d), preferably at about 4° C. One way to identify the dendritic cell-enriched fraction is by fluorescence-activated cell sorting. The white blood cell fraction can be treated with calcium ionophore in the presence of other cytokines, such as recombinant (rh) rhIL-12, rhGM-CSF, or rhIL-4. The cells of the white blood cell fraction can be washed in buffer and suspended in Ca⁺⁺/Mg⁺⁺ free media prior to the separating step. The white blood cell fraction can be obtained by leukopheresis. The dendritic cells can be identified by the presence of at least one of the following markers: HLA-DR, HLA-DQ, or B7. 2, and the simultaneous absence of the following markers: CD3, CD14, CD16, 56, 57, and CD 19, 20. Monoclonal antibodies specific to these cell surface markers are commercially available.

More specifically, the method requires collecting an enriched collection of white cells and platelets from leukopheresis that is then further fractionated by countercurrent centrifugal elutriation (CCE). Abrahamsen et al. (1991) J. Clin. Apheresis. 6:48-53. Cell samples are placed in a special elutriation rotor. The rotor is then spun at a constant speed of, for example, 3000 rpm. Once the rotor has reached the desired speed, pressurized air is used to control the flow rate of cells. Cells in the elutriator are subjected to simultaneous centrifugation and a washout stream of buffer which is constantly increasing in flow rate. This results in fractional cell separations based largely but not exclusively on differences in cell size.

Quality control of APC and more specifically DC collection and confirmation of their successful activation in culture is dependent upon a simultaneous multi-color FACS analysis technique which monitors both monocytes and the dendritic cell subpopulation as well as possible contaminant T lymphocytes. It is based upon the fact that DCs do not express the following markers: CD3 (T cell); CD14 (monocyte); CD16, 56, 57 (NK/LAK cells); CD19, 20 (B cells). At the same time, DCs do express large quantities of HLA-DR, significant HLA-DQ and B7.2 (but little or no B7.1) at the time they are circulating in the blood (in addition they express Leu M7 and M9, myeloid markers which are also expressed by monocytes and neutrophils).

Once collected, the DC rich/monocyte APC fractions (usually 150 through 190) can be pooled and cryopreserved for future use, or immediately placed in short term culture.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 24-48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2.

Specific combination(s) of cytokines have been used successfully to amplify (or partially substitute) for the activation/conversion achieved with calcium ionophore: these cytokines include but are not limited to purified or recombinant human (“rh”) rhGM-CSF, rhIL-2, and rhIL-4. Each cytokine when given alone is inadequate for optimal upregulation.

Immune Effector Cells

The present invention makes use of the above-described antigen-presenting cells to stimulate production of an enriched population of antigen-specific immune effector cells. Accordingly, the present invention provides a population of cells enriched in educated, antigen-specific immune effector cells, specific for an antigenic peptide of the invention. These cells can cross-react with (bind specifically to) antigenic determinants (epitopes) on antigens. In some embodiments, the antigen is on the surface of tumor cells and the educated, antigen-specific immune effector cells of the invention suppress growth of the tumor cells. When APCs are used, the antigen-specific immune effector cells are expanded at the expense of the APCs, which die in the culture. The process by which naive immune effector cells become educated by other cells is described essentially in Coulie (1997) Molec. Med. Today 3:261-268.

The APCs prepared as described above are mixed with naive immune effector cells. Preferably, the cells may be cultured in the presence of a cytokine, for example IL2. Because dendritic cells secrete potent immunostimulatory cytokines, such as IL-12, it may not be necessary to add supplemental cytokines during the first and successive rounds of expansion. In any event, the culture conditions are such that the antigen-specific immune effector cells expand (i.e. proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding. Methods for introducing transgenes in vitro, ex vivo and in vivo are well known in the art. See Sambrook, et al. (1989) supra.

An effector cell population suitable for use in the methods of the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69⁺ cells, or inactivating alloreactive cells, or inducing anergy in the alloreactive cell population.

Hybrid immune effector cells can also be used. Immune effector cell hybrids are known in the art and have been described in various publications. See, for example, International Patent Application Nos. WO 98/46785; and WO 95/16775.

The effector cell population can comprise unseparated cells, i.e., a mixed population, for example, a PBMC population, whole blood, and the like. The effector cell population can be manipulated by positive selection based on expression of cell surface markers, negative selection based on expression of cell surface markers, stimulation with one or more antigens in vitro or in vivo, treatment with one or more biological modifiers in vitro or in vivo, subtractive stimulation with one or more antigens or biological modifiers, or a combination of any or all of these.

Effector cells can obtained from a variety of sources, including but not limited to, PBMC, whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include an immunized donor, a non-immunized (naïve) donor, treated or untreated donors. A “treated” donor is one that has been exposed to one or more biological modifiers. An “untreated” donor has not been exposed to one or more biological modifiers.

Methods of extracting and culturing effector cells are well known. For example, effector cells can be obtained by leukapheresis, mechanical apheresis using a continuous flow cell separator. For example, lymphocytes and monocytes can be isolated from the buffy coat by any known method, including, but not limited to, separation over Ficoll-Hypaque™ gradient, separation over a Percoll gradient, or elutriation. The concentration of Ficoll-Hypaque™ can be adjusted to obtain the desired population, for example, a population enriched in T cells. Other methods based on affinity are known and can be used. These include, for example, fluorescence-activated cell sorting (FACS), cell adhesion, magnetic bead separation, and the like. Affinity-based methods may utilize antibodies, or portions thereof, which are specific for cell-surface markers and which are available from a variety of commercial sources, including, the American Type Culture Collection (Manassas, Md.). Affinity-based methods can alternatively utilize ligands or ligand analogs, of cell surface receptors.

The effector cell population can be subjected to one or more separation protocols based on the expression of cell surface markers. For example, the cells can be subjected to positive selection on the basis of expression of one or more cell surface polypeptides, including, but not limited to, “cluster of differentiation” cell surface markers such as CD2, CD3, CD4, CD8, TCR, CD45, CD45RO, CD45RA, CD11b, CD26, CD27, CD28 , CD29, CD30, CD31, CD40L; other markers associated with lymphocyte activation, such as the lymphocyte activation gene 3 product (LAG3), signaling lymphocyte activation molecule (SLAM), T1/ST2; chemokine receptors such as CCR3, CCR4, CXCR3, CCR5; homing receptors such as CD62L, CD44, CLA, CD146, a4b7, aEb7; activation markers such as CD25, CD69 and OX40; and lipoglycans presented by CD1. The effector cell population can be subjected to negative selection for depletion of non-T cells and/or particular T cell subsets. Negative selection can be performed on the basis of cell surface expression of a variety of molecules, including, but not limited to, B cell markers such as CD19, and CD20; monocyte marker CD14; the NK cell marker CD56.

An effector cell population can be manipulated by exposure, in vivo or in vitro, to one or more biological modifiers. Suitable biological modifiers include, but are not limited to, cytokines such as IL-2, IL-4, IL-10, TNF, IL-12, IFN; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL2 receptor, anti-CD28 ; chemokines, including, for example, lymphotactin. The biological modifiers can be native factors obtained from natural sources, factors produced by recombinant DNA technology, chemically synthesized polypeptides or other molecules, or any derivative having the functional activity of the native factor. If more than one biological modifier is used, the exposure can be simultaneous or sequential.

The present invention provides compositions comprising immune effector cells, which may be T cells, enriched in antigen-specific cells, specific for a peptide of the invention. By “enriched” is meant that a cell population is at least about 50-fold, more preferably at least about 500-fold, and even more preferably at least about 5000-fold or more enriched from an original naive cell population. The proportion of the enriched cell population which comprises antigen-specific cells can vary substantially, from less than 10% up to 100% antigen-specific cells. If the cell population comprises at least 50%, preferably at least 70%, more preferably at least 80%, and even more preferably at least 90%, antigen-specific immune effector cells, specific for a peptide of the invention, then the population is said to be “substantially pure”. The percentage which are antigen-specific can readily be determined, for example, by a ³H-thymidine uptake assay in which the effector cell population (for example, a T-cell population) is challenged by an antigen-presenting matrix presenting an antigenic peptide of the invention.

Compositions of the Invention

This invention also provides compositions containing any of the above-mentioned recombinant polynucleotides, gene delivery vehicles, host cells, including but not limited to antigen presenting cells, or educated immune effector cells, and an acceptable solid or liquid carrier. When the compositions are used pharmaceutically, they are combined with a “pharmaceutically acceptable carrier” for diagnostic and therapeutic use. These compositions also can be used for the preparation of medicaments for the diagnostic and immunomodulatory methods of the invention.

Vaccines for Cancer Treatment and Prevention

This invention provides vaccines for cancer treatment and prevention. In addition to the previously characterized tumor antigens, cancer cells contain many new antigens potentially recognizable by the immune system. Given the speed with which epitopes can be identified, custom anticancer vaccines can be generated for affected individuals by isolating TILs from patients with solid tumors, determining their MHC restriction, and assaying these CTLs against the appropriate library for reactive epitopes. These vaccines will be both treatments for affected individuals as well as preventive therapy against recurrence (or establishment of the disease in patients which present with a familial genetic predisposition to it). Inoculation of individuals who have never had the cancer is expected to be quite successful as preventive therapy, even though a tumor antigen-specific CTL response has not yet been elicited, because in most cases high affinity peptides seem to be immunogenic suggesting that holes in the functional T cell repertoire, if they exist, may be relatively rare. Sette et al. (1994) J. Immunol., 153:5586-5592. In mice, vaccination with appropriate epitopes not only eliminates established tumors but also protects against tumor re-establishment after inoculation with otherwise lethal doses of tumor cells. Bystryn et al. (1993) Supra.

Vaccines for Diseases Caused by Pathogenic Organisms

The recombinant polynucleotides containing epitopes that induce an immune response to pathogens, as well as vectors and host cells containing them, also are useful in methods to induce (or increase, or enhance) an immune response to a pathogenic organism. These include pathogenic viruses, bacteria, and protozoans.

Viral infections are ideal candidates for immunotherapy. Immunological responses to viral pathogens are sometimes ineffective as in the case of the lentiviruses such as HIV which causes AIDS. The high rates of spontaneous mutation make these viruses elusive to the immune system. However, a saturating profile of CTL epitopes presented on infected cells will identify shared antigens among different serotypes in essential genes that are largely intolerant to mutation which would allow the design of more effective vaccines.

Diagnostic, Prognostic and Therapeutic Utilities

The present invention provides diagnostic and immunomodulatory methods using the recombinant polynucleotides, gene delivery vehicles containing the polynucleotides, cells (including APCs and educated immune effector cells), i.e., immunomodulatory agents, of the invention.

Diagnostic Methods

The present invention provides diagnostic methods using the compositions described above. The cells expressing the epitopes in the context of an MHC molecule can be used to detect and monitor the presence of an antigen-specific CD4⁺ or CD8⁺ T cell which binds the epitope or epitopes expressed by the recombinant polynucleotide. It also can be used to determine which antigenic peptide and/or the number of epitopes are optimal therapeutic candidate to induce a T cell response, to induce T cell anergy or to educate antigen specific immune effector cells. (See, for example, the experimental section below.) This also provides a simple assay to detect the optimal epitope for each patient when more than one epitope or antigen is deemed appropriate for a particular disease. For example there exist several well characterized melanoma antigens and the optimal epitope will likely vary with each patient being therapeutically or prophylactically treated.

The diagnostic methods of the invention also include: (1) assays to predict the in vivo efficacy of a recombinant polynucleotide of the invention; (2) assays to determine the precursor frequency (i.e., the presence and number of) of immune effector cells specific for an antigenic peptide produced by a recombinant polynucleotide of the invention; and (3) assays to monitor the efficacy of a recombinant polynucleotide of the invention once it has been used in an immunomodulatory method of the invention.

Diagnostic methods of the invention are generally carried out under suitable conditions and for a sufficient time to allow specific binding to occur between the expressed antigenic peptide of the invention and an immune effector molecule, such as a TCR, on the surface of an immune effector cell, such as a CD4+ or CD8+ T cell. “Suitable conditions” and “sufficient time” are generally conditions and times suitable for specific binding. Suitable conditions occur between about 4° C. and about 40° C., preferably between about 4° C. and about 37° C., in a buffered solution, and within a pH range of between 5 and 9. A variety of buffered solutions are known in the art, can be used in the diagnostic methods of this invention, and include, but are not limited to, phosphate-buffered saline. Sufficient time for binding and response will generally be between about 1 second and about 24 hours after exposure of the sample to the antigenic peptide epitope of the invention.

In some embodiments, the invention provides diagnostic assays to predict the efficacy of an antigenic peptide of the invention. In some of these embodiments, defined T cell epitopes are used to clinically characterize tumors and viral pathogens to determine in advance, the predicted efficacy of an in vivo vaccine trial. This can be achieved by a simple proliferation assay of a patient's peripheral blood mononuclear cells. Recombinant polynucleotides encoding peptides which elicit a response are viable vaccine candidates for that patient.

Immunomodulatory Methods

Also provided are methods of modulating an immune response in a subject by administering an effective amount of a composition of this invention. Immunomodulatory methods of the invention include methods that result in induction or increase, as well as methods that result in suppression or reduction, of an immune response in a subject, and comprise administering to the subject an effective amount of a composition of the invention (recombinant polynucleotide, host cell or immune effector cell, and any combination thereof) under conditions that result in the desired effect on an immune response (or lack thereof) to the peptide. Immunomodulatory methods of the invention include vaccine methods, adoptive immunotherapy, and methods to induce T cell unresponsiveness or anergy.

The recombinant polynucleotides of the invention can be administered as naked DNA or in a gene delivery vehicle. Alternatively, host cells that comprise the recombinant polynucleotide are administered to the subject. Still further, immune effector cells that have been educated in the presence and at the expense of host cells presenting antigen are administered in an effective amount to the subject. These compositions can be combined with appropriate and effective amount of an adjuvant, cytokine or co-stimulatory molecule for an effective vaccine regimen. In some embodiments, the host cell is an APC, such as a dendritic cell. The host cell can be further modified by inserting of a polynucleotide coding for an effective amount of either or both of a cytokine a co-stimulatory molecule.

One can determine if the immune response has been altered, enhanced or suppressed by comparing T cell activation prior to and after therapy. Various methods are known to evaluate T cell activation. CTL activation can be detected by any known method, including but not limited to, tritiated thymidine incorporation (indicative of DNA synthesis), and examination of the population for growth or proliferation, e.g., by identification of colonies. Alternatively, the tetrazolium salt MTT (3-(4,5-dimethyl-thazol-2-yl)-2,5-diphenyl tetrazolium bromide) may be added. Mossman (1983) J. Immunol. Methods 65:55-63; Niks and Otto (1990) J. Immunol. Methods 130:140-151. Succinate dehydrogenase, found in mitochondria of viable cells, converts the MTT to formazan blue. Thus, concentrated blue color would indicate metabolically active cells. In yet another embodiment, incorporation of radiolabel, e.g., tritiated thymidine, may be assayed to indicate proliferation of cells. Similarly, protein synthesis may be shown by incorporation of ³⁵S-methionine. In still another embodiment, cytotoxicity and cell killing assays, such as the classical chromium release assay, may be employed to evaluate epitope-specific CTL activation. To detect activation of CD4+ T cells, any of a variety of methods can be used, including, but not limited to, measuring cytokine production; and proliferation, for example, by tritiated thymidine incorporation

Release of ⁵¹Cr from labeled target cells is a standard assay which can be used to assess the number of peptide-specific CTLs in a biological sample. Tumor cells, or APCs of the invention, are radiolabeled as targets with about 200 μCi of Na₂ ⁵¹CrO₄ for 60 minutes at 37° C., followed by washing. T cells and target cells (˜1×10⁴/well) are then combined at various effector-to-target ratios in 96-well, U-bottom plates. The plates are centrifuged at 100×g for 5 minutes to initiate cell contact, and are incubated for 4-16 hours at 37° C. with 5% CO₂. Release of ⁵¹Cr is determined in the supernatant, and compared with targets incubated in the absence of T cells (negative control) or with 0.1% TRITON™ X-100 (positive control). See, e.g., Mishell and Shiigi, eds. Selected Methods in Cellular Immunology (1980) W. H. Freeman and Co.

The methods of this invention can be further modified by co-administering an effective amount of a cytokine or co-stimulatory molecule to the subject. These methods can be further modified by combination with any previously known therapy, e.g., chemotherapy and radiation therapy for the treatment of cancer and co-administration of anti-viral drugs to counter an infectious disease.

The agents provided herein as effective for their intended purpose can be administered to subjects having a disease to be treated with an immunomodulatory method of the invention or to individuals susceptible to or at risk of developing such a disease. When the agent is administered to a subject such as a mouse, a rat or a human patient, the agent can be added to a pharmaceutically acceptable carrier and systemically or topically administered to the subject. Therapeutic amounts can be empirically determined and will vary with the pathology or condition being treated, the subject being treated and the efficacy and toxicity of the therapy.

The amount of a polynucleotide, host cell or immune effector cell administered to the subject will vary depending, in part, on its intended effect, and is ultimately at the discretion of the medical or veterinary practitioner. The factors to be considered include the condition being treated, the route of administration, and nature of the formulation, the subject's body weight, surface area, age, and general condition and the particular peptide to be administered. A suitable effective dose of peptides of the invention generally lies in the range of from about 0.0001 μmol/kg to about 1000 μmol/kg bodyweight. The total dose may be given as a single dose or multiple doses, e.g., two to six times per day. For example, for a 75 kg mammal (e.g., a human) the dose range would be about 2.25 μmol/kg/day and a typical dose could be about 100 μmol of peptide. If discrete multiple doses are indicated treatment might typically be 25 μmol of a peptide of the invention given up to 4 times per day. In an alternative administrative regimen, peptides of the invention may be given on alternate days or even once or twice a week. A suitable effective dose of an immune effector cell of the invention generally lies in the range of from about 10² to about 10⁹ cells per administration. Cells can be administered once, followed by monitoring of the clinical response, such as diminution of disease symptoms or tumor mass. Administration may be repeated on a monthly basis, for example, or as appropriate. Those skilled in the art will appreciate that an appropriate administrative regimen would be at the discretion of the physician or veterinary practitioner.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including nasal, topical (including transdermal, aerosol, buccal and sublingual), parenteral (including subcutaneous, intramuscular, intravenous and intradermal) and pulmonary. It will also be appreciated that the preferred route will vary with the condition and age of the recipient, and the disease or condition being treated.

Adoptive Immunotherapy Methods

The expanded populations of antigen-specific immune effector cells and APCs of the present invention find use in adoptive immunotherapy regimes and as vaccines.

Adoptive immunotherapy methods involve, in one aspect, administering to a subject a substantially pure population of educated, antigen-specific immune effector cells made by culturing naive immune effector cells with APCs as described above. In some embodiments, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells comprising the recombinant polynucleotide of this invention, is administered to the same patient.

In a further embodiment, APCs or immune effector cells are administered with an effective amount of a stimulatory cytokine, such as IL-2 or a co-stimulatory molecule.

Methods of Inducing T Cell Anergy

The recombiant polynucleotides of this invention are useful in methods to induce T cell unresponsiveness, or anergy. Disorders which can be treated using these methods include autoimmune disorders, allergies, and allograft rejection.

Autoimmune disorders are diseases in which the body's immune system responds against self tissues. They include most forms of arthritis, ulcerative colitis, and multiple sclerosis. Synthetic antigenic peptide epitopes corresponding to endogenous elements that are recognized as foreign can be used in the development of treatments using gene therapy or other approaches. For example, synthetic CTL epitopes, which can act as “suicide substrates” for CTLs that mediate autoimmunity, can be designed as described above. That is to say, peptides which have a high affinity for the MHC allele but fail to activate the TCR could effectively mask the cellular immune response against cells presenting the antigen in question. In support of this approach, it is believed that the long latency period of the HIV virus is due to an antiviral immune response and a mechanism by which the virus finally evades the immune system is by generating epitopes that occupy the MHC molecules but do not stimulate a TCR lytic response, inducing specific T cell anergy. Klenerman et al. (1995) Eur. J. Immunol., 25:1927-1931.

In vitro stimulation of T cells through the complex of T cell-antigen receptor and CD3 alone in the absence of other signals, induces T cell anergy or paralysis. T cell activation as measured by interleukin-2 production and proliferation in vitro requires both antigenic and co-stimulatory signals engendered by cell to cell interactions among antigen-specific T cells and antigen presenting cells. Various interactions of these CD2 proteins on the T-cell surface with CD58 (LFA-3) proteins and antigen-presenting cells, those of CD11aCD18 (LFA-1) proteins with CD54 (ICAM-1) proteins and those of CD5 proteins with CD72 proteins can impart such a co-stimulatory signal in vitro. Cytokines derived from antigen-presenting cells (e.g., interleukin-1 and interleukin-6) can also provide co-stimulatory signals that result in T-cell activation in vitro. The delivery of both antigenic and co-stimulatory signals leads to stable transcription of the interleukin-2 gene and other pivotal T cell-activation genes. The foregoing co-stimulatory signals depend on protein kinase C and calcium. Potent antigen presenting cells express CD80 (B7 and BB1) and other related surface proteins and many T cells express B7 binding proteins, namely CD28 and CTLA-4 proteins. Binding of CD80 by CD28 and CDLA-4 stimulates a T cell co-stimulatory pathway that is independent of protein kinase C and calcium leading to vigorous T cell proliferation. The stimulation of B cells also depends on the interaction between the specific antigen and the cell-surface immunoglobulin. T cell derived cytokines (e.g., interleukins 1 and 4), physical contact between T cells and B cells through specific pairs of receptors and co-receptors, or both, provide the signal or signals essential for B cell stimulation.

Conventional routes of administration are used. A T-cell stimulating or anergy producing amount (or therapeutically effective amount as described above) of an immunotherapeutic antigen-superantigen polymer according to the invention is contacted with the target cells. By “T-cell anergy effective amount” is intended an amount which is effective in producing a statistically significant inhibition of a cellular activity mediated by a TCR. This may be assessed in vitro using T-cell activation tests. Typically, T-cell anergy or activation is assayed by tritiated thymidine incorporation in response to specific antigen.

To determine whether anergy has been induced, the T cells to be tested can be cultured together with an antigen presenting matrix which presents the epitope or epitopes expressed by the recombinant polynucleotide of the invention in an MHC Class I or Class II molecule together with co-stimulatory molecules necessary to activate the T cell. The cultures are incubated for about 48 hours, then pulsed with tritiated thymidine and incorporation measured about 18 hours later. The absence of incorporation above control levels, where the T-cells are presented with antigen presenting cells which do not stimulate the T cells, either due to using an MHC to which the T cells are not restricted or using a peptide to which the T cells are not sensitive, is indicative of an absence of activation. One may use other conventional assays to determine the extent of activation, such as assaying for IL-2, -3, or -4, cell surface proteins associated with activation, e.g. CD71 or other convenient techniques. Another method is to determine the expression of a protein which is expressed on quiescent T cells, but not on anergic T cells. See, e.g., U.S. Pat. No. 5,747,299.

Therapeutic and Prophylactic Administrations

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally, transdermally or by inhalation therapy, and may take the form of tablets, lozenges, granules, capsules, pills, ampoules, suppositories or aerosol form. They may also take the form of gene therapy, suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

The following examples are intended to illustrate, but not limit this invention.

### EXAMPLES

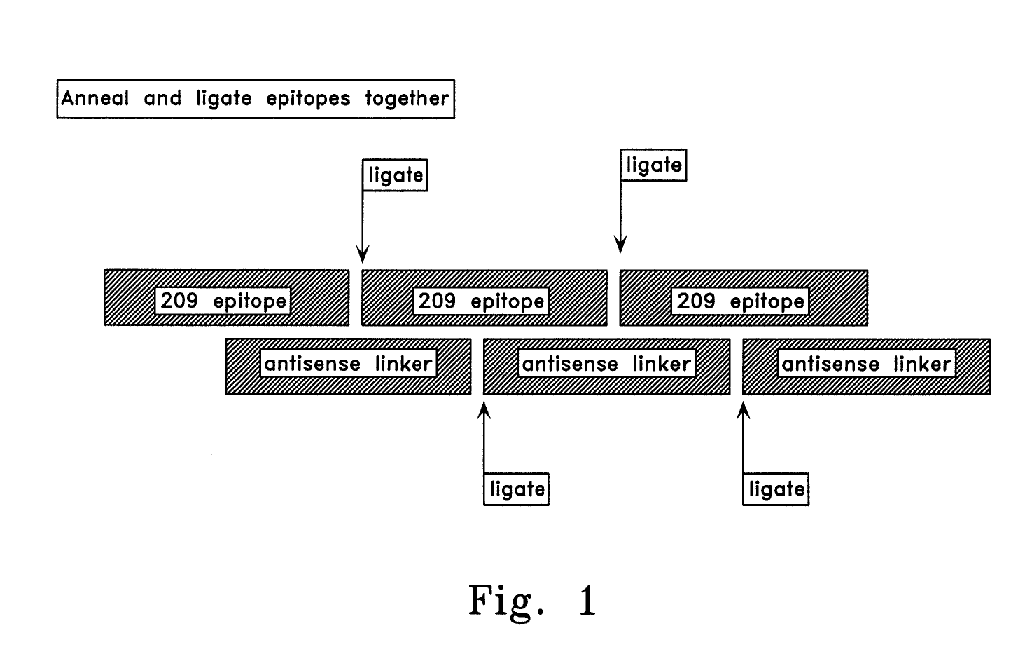

A synthetic double stranded DNA sequence as shown in FIG. 1 encodes the 209 epitope of the human melanoma antigen gp100. It is engineered to have cohesive ends such that it can be ligated together to form head to tail concatamers. A DNA sequence encoding a methionine residue required for translational initiation is appended to the 5′ end of the concatamer and the entire sequence is placed under the control of a strong transcriptional promoter (such as CMV) within a DNA vector (such as an adenovirus). The DNA vector is introduced into the target cells where transcription and subsequent translation of the mini-gene gives rise to a polypeptide consisting of repeats of the gp100 209 peptide.

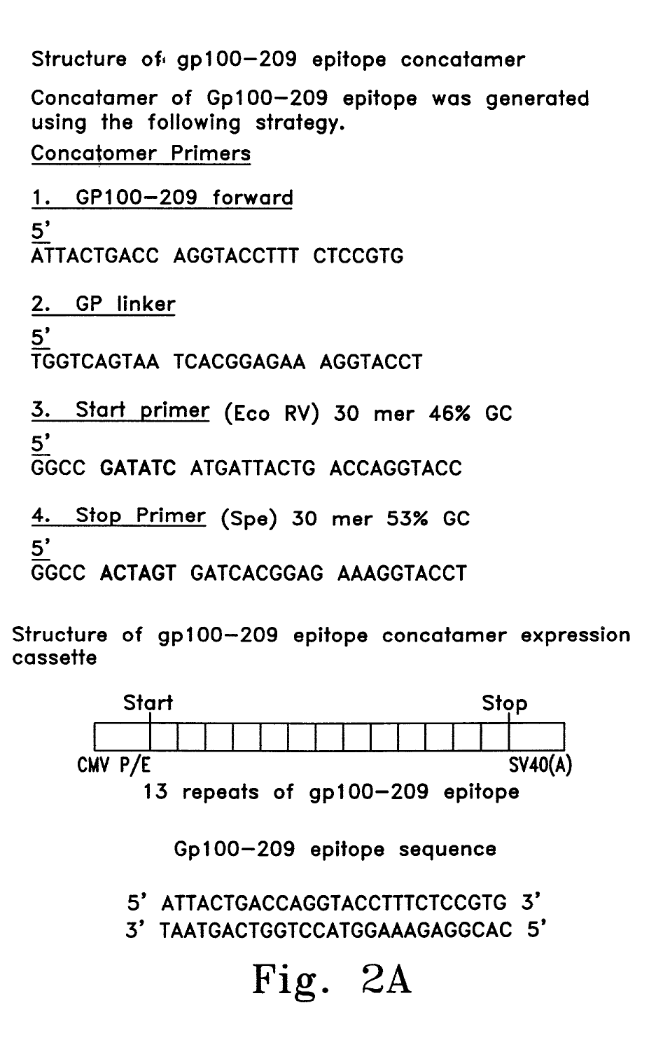

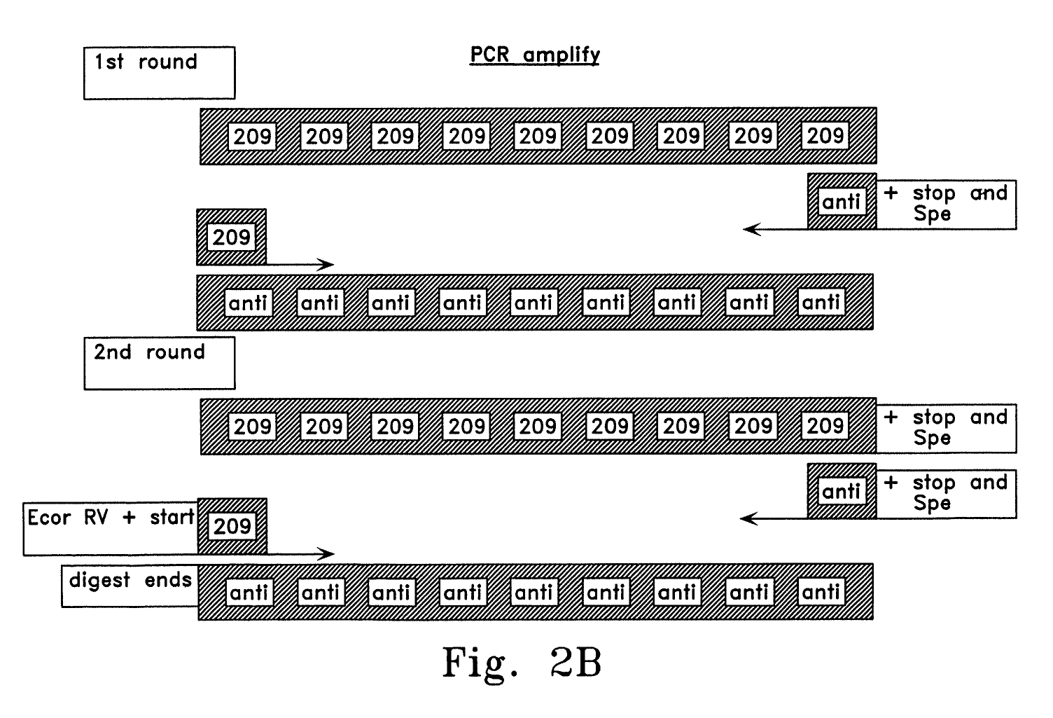

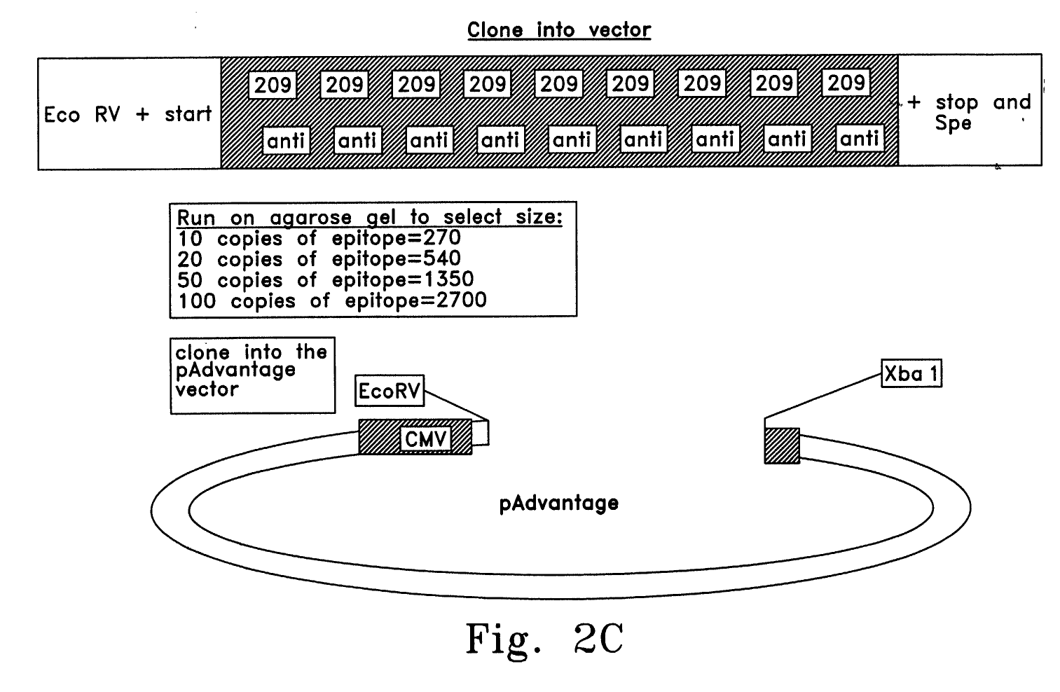

FIG. 2A shows the primers used to construct the gp100 209 multimers used in the construction of the recombinant polynucleotide. One method to make the recombinant vectors applies a modification of the methods disclosed in Toes et al. (1997) PNAS USA 94:14660-14665. Briefly, melanoma gp 100 209 forward and linker primers were first annealed and ligated. The ligated products were PCR amplified using start and stop primers to generate concatamers of varying number of gp 100- epitopes. PCR products were digested with EcorV and Spe-1 and cloned into pSV₂-iceu1 adenovirus shuttle vector and sequenced. The clone with the correct sequence of 13 repeats of gp 100 209 epitope was selected and used for generating recombinant viral vectors. The method was repeated and recombinant polynucleotides encoding varying repeats were generated.

FIG. 2B shows sequence and restriction enzyme sites that are useful for an alternative embodiment of this invention.

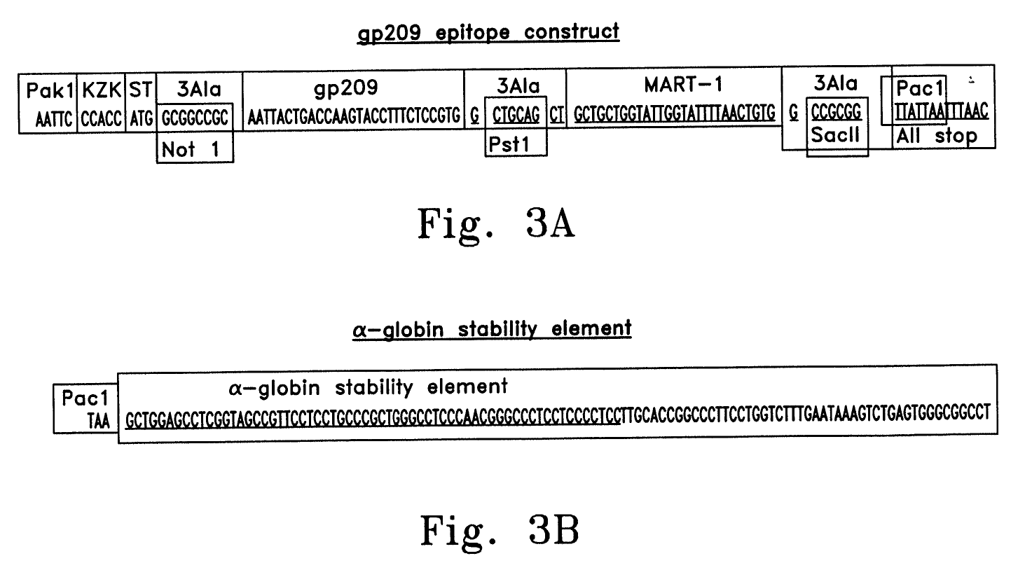

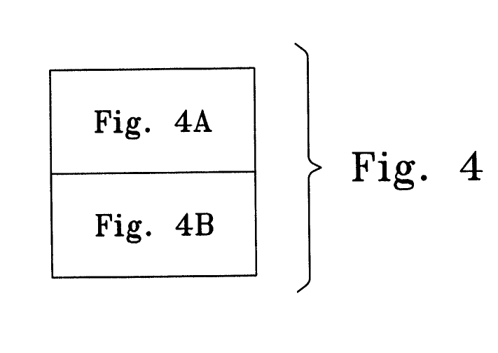

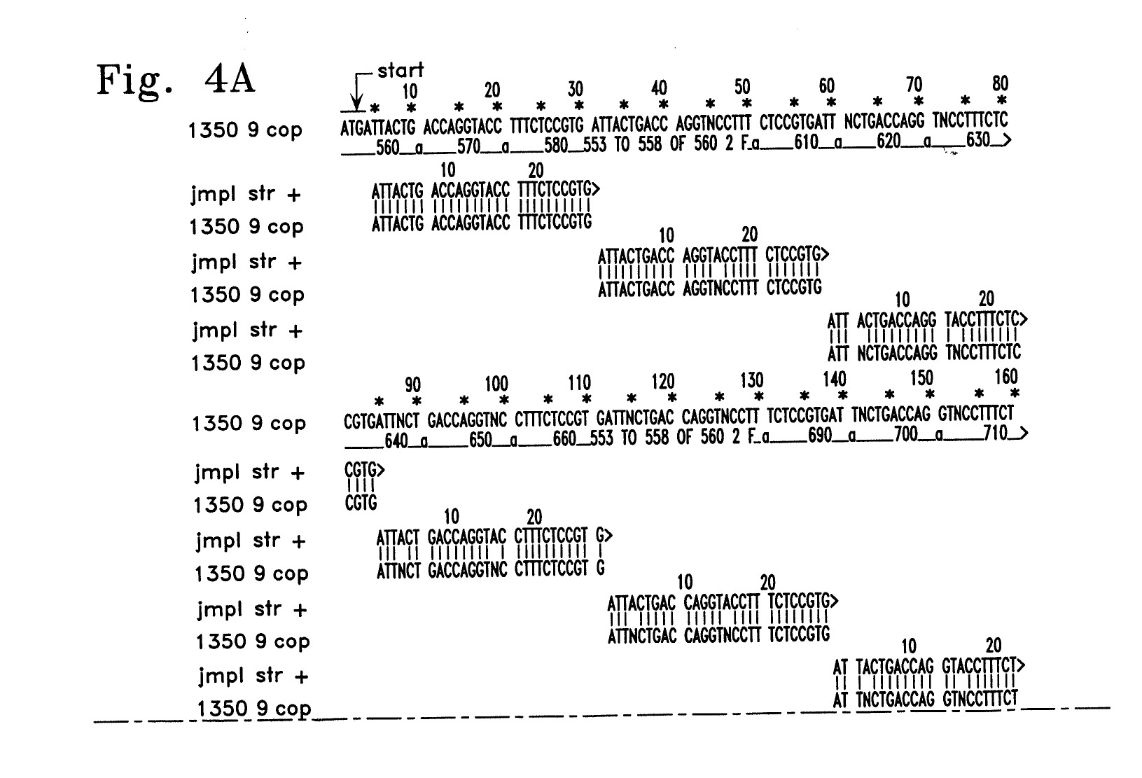

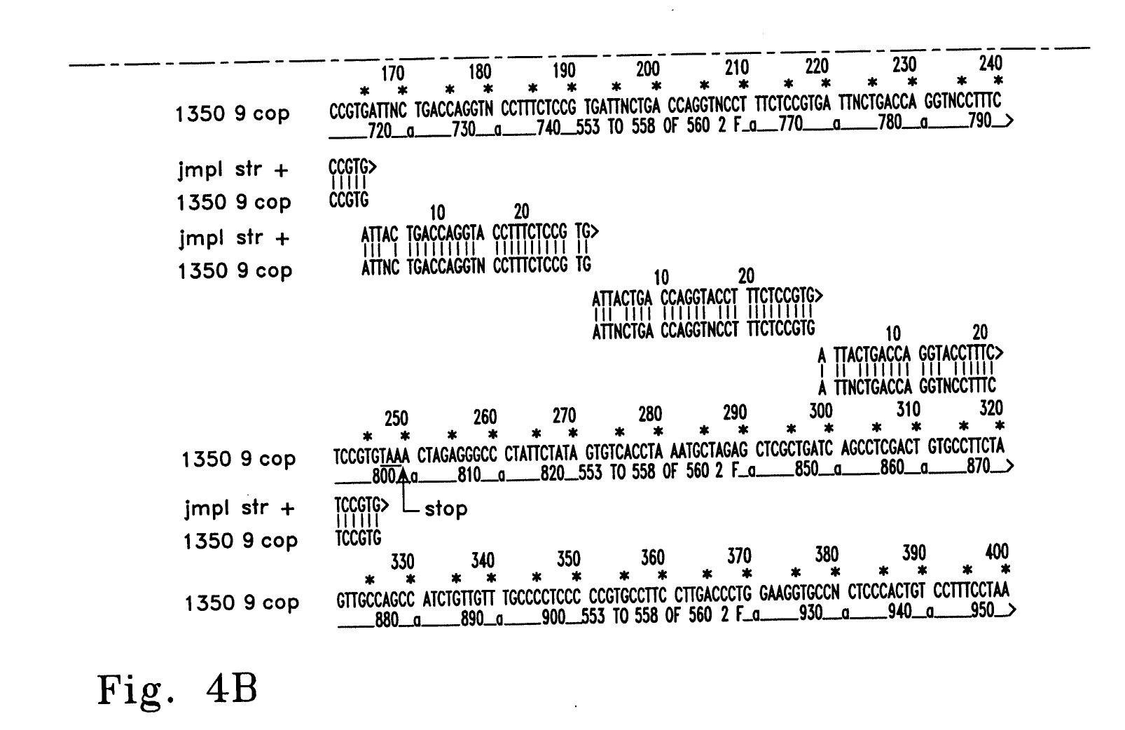

FIG. 2C shows a further embodiment of the invention wherein the polynucleotide further comprises a polynucleotide corresponding to an mRNA stability element, e.g., the 3′UTR of human α-globin (Holick and Liebhaber (1997) PNAS USA 94:2410-2414) or the 3′UTR of murine α-globin (Wang and Liebhaber (1996) EMO J. 15(18):5040-5051) or their functional equivalents. FIG. 3A schematically shows a further embodiment of the invention wherein the antigenic peptides are flanked by 3 alanine residues which act as a buffer to assist in the proper processing of the epitope, but also provide space for easy manipulation of the construct.

In a further embodiment, the polynucleotide comprises multiple copies of the antigenic epitope and a polycucleotide coding for a viral internal ribosome entry site (IRES) using a modification of the method disclosed in U.S. Pat. No. 5,770,428.

In order to confirm for the ability of vector encoding multiple copies of the epitope to present antigen more efficiently a recombinant adenovirus encoding multiple copies of 209 epitope was constructed, and a CTL assay was carried out using the MDA 231 breast adenocarcinoma cell line (available from ATCC, catalogue number HTB-26) that were infected with recombinant adenovirus virus encoding gp 100 sequence (single epitope) or the concatamer (with 13 copies of 209 epitope sequence) as targets by reacting with Hurley's T cells that recognize gp 100-209 epitope in HLA-A2 restricted manner.

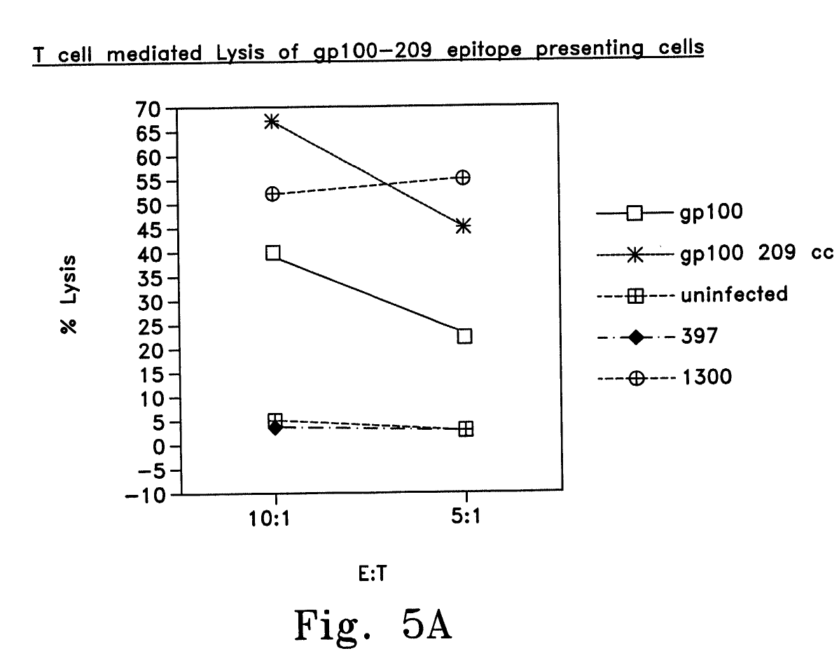

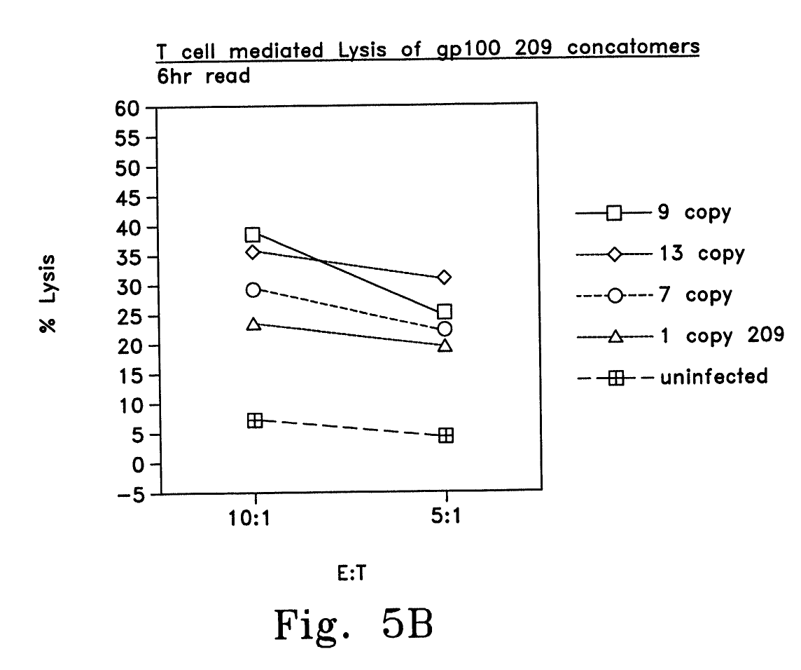

FIG. 5 graphically shows the results of the CTL assay. Uninfected MDA 231 cells, MDA 231 cells that were infected with recombinant adenovirus encoding gp 100 sequence (gp 100) or the concatamer (gp 100-209 cc), positive control melanoma cell line expresses gp100 but not HLA-A2 (397) were used in chromium release CTL assay and percent lysis was calculated and plotted in the graph shown in FIG. 5A. FIG. 5B graphically compares the results of the lysis assay with varying copies of the epitope.

These results indicate that cells that were infected with virus encoding the concatamer are lysed much more (almost two fold) efficiently than cells that were infected with virus encoding a single epitope indicating that there is more potent antigen presentation by cells that express multiple copies of the epitope.

Even the positive control cells that express high levels of gp 100 and HLA-A2 molecule are lysed equivalently or slightly less efficiently than gp100-209 expressing cells indicating that the presentation of the epitope in cells that express concatamer is comparable or more efficient than melanoma tumor cells that express high levels of gp100 (See FIG. 5A). The negative controls, Mel-397, which express high levels of gp100 but not HLA-A2 and uninfected MDA231 cells are not lysed indicating the specificity of T cells to recognize and lyse cells that express gp100-209 epitope and HLA-A2 molecules.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

What is claimed is:

1. A recombinant polynucleotide comprising a plurality of first polynucleotides encoding an identical antigenic peptide and wherein the first polynucleotides are operatively linked to each other to enhance translation of the polynucleotides to the antigenic peptide and binding of the antigenic peptide to MHC molecules.

2. The recombinant polynucleotide of claim 1, further composing a plurality of a second polynucleotide encoding multiple copies of an antigenic peptide having an amino acid sequence that is different from the peptides encoded by the first polynucleotides.

3. The recombinant polynucleotide of claim 1, wherein the plurality of first polynucleotides is 2 or more.

4. The recombinant polynucleotide of claim 1, wherein the plurality of first polynucleotides is 7 or more.

5. The recombinant polynucleotide of claim 1, wherein the plurality of first polynucleotides is 9 or more.

6. The recombinant polynucleotide of claim 1, wherein the plurality of first polynucleotides is 13 or more.

7. The recombinant polynucleotide of claim 2, wherein the plurality of second polynucleotides is 2 or more.

8. The recombinant polynucleotide of claim 2, wherein the plurality of second polynucleotides is 7 or more.

9. The recombinant polynucleotide of claim 2, wherein the plurality of second polynucleotides is 9 or more.

10. The recombinant polynucleotide of claim 2, wherein the plurality of second polynucleotides is 13 or more.

11. The recombinant polynucleotide of claims 1-10, further comprising a promoter operatively linked to the polynucleotide.

12. The recombinant polynucleotide of claims 1-10, further comprising a polynucleotide encoding a cytokine.

13. The recombinant polynucleotide of claims 1-10, further comprising a polynucleotide encoding a costimulatory molecule.

14. The recombinant polynucleotide of claims 1-10, further comprising a polynucleotide encoding a cytokine and a polynucleotide encoding a co-stimulatory molecule.

15. The recombinant polynucleotide of claims 1-10, further comprising a polynucleotide encoding a plurality of amino acids inserted between the plurality of polynucleotides encoding the antigenic peptides.

16. The recombinant polynucleotide of claim 15, wherein the plurality of amino acids is at least three alanines.

17. The recombinant polynucleotide of claims 1-10, further comprising a polynucleotide having mRNA stability activity operatively linked to the polynucleotides encoding the antigenic peptides to stabilize the mRNA transcribed from the recombinant polynucleotide.

18. The polynucleotide of claim 12, wherein the polynucleotide having mRNA stability activity is the 3′ UTR of the α-globulin gene.

19. The recombinant polynucleotide of claims 1-10, wherein the antigenic peptide is an antigenic fragment of a tumor associated antigen.

20. The recombinant polynucleotide of claim 19, wherein the tumor associated antigen is selected from the group consisting of melanoma gp 100, MART-1, melan-A, tyrosinase, TRP-1, TRP-2, Her-2, Muc-1, and CEA.

21. The recombinant polynucleotide of claim 19, wherein the tumor associated antigen is gp 100.

22. The recombinant polynucleotide of claim 19, wherein the antigenic fragment of a tumor associated antigen is gp 209 of gp 100.

23. The recombinant polynucleotide of claims 1-10, wherein the antigenic peptide is a fragment of pathogenic antigen.

24. The recombinant polynucleotide of claims 23, wherein the pathogen is a bacteria or virus.

25. A gene delivery vehicle comprising the recombinant polynucleotide of claims 1-10.

26. The gene delivery vehicle of claim 25, wherein the vehicle is selected from the group consisting of a viral vector, a liposome, and a plasmid.

27. A gene delivery vehicle comprising the recombinant polynucleotide of claim 11.

28. The gene delivery vehicle of claim 27, wherein the vehicle is selected from the group consisting of a viral vector, a liposome, and a plasmid.

29. A host cell comprising the recombinant polynucleotide of claims 1-10.

30. The host cell of claim 29, wherein the host cell is a eucaryotic or a procaryotic cell.

31. The host cell of claim 29, wherein the cell is a dendritic cell.

32. The host cell of claim 29 or 31, wherein the cell is a mammalian cell.

33. The host cell of claim 32, wherein the mammalian cell is a human cell.

34. The host cell of claim 31, wherein the dendritic cell is an antigen presenting cell.

35. A method for presenting antigenic epitopes on the surface of an antigen presenting cell comprising introducing the recombinant polynucleotide of any of claims 1-10 under suitable conditions such that the polynucleotide encoding the antigenic peptide is translated and presented on the surface of the antigen presenting cell.

36. A method for generating educated, immune effector cells comprising culturing the host cells of claim 34 with naive immune effector cells under conditions such that the immune effector cells proliferate at the expense of the host cells.

37. An educated immune effector cell, which has been, cultured in the presence and at the expense of the cells of claim 34.

38. A method of modulating an immune response in a subject, comprising administering to the subject an effective amount of the polynucleotide of claims 1-10.

39. A method of modulating an immune response in a subject, comprising administering to the subject an effective amount of the host cell of claim 34.

40. A method of modulating an immune response in a subject, comprising administering to the subject an effective amount of the immune effector cell of claim 37.

41. The method of claim 38, further comprising administering an effective amount of a cytokine and/or costimulatory molecule to the subject.

42. The method of claim 39, further comprising administering an effective amount of a cytokine and/or costimulatory molecule to the subject.

43. The method of claim 40, further comprising administering an effective amount of a cytokine and/or costimulatory molecule to the subject.
